(19) United States
(12) Patent Application Publication
(10) Pub. No.: US 2008/0048541 A1
(43) Pub. Date: Feb. 28, 2008

(54) POLYMER-THERMAL SHIELD FOR ULTRA-VIOLET LAMP

(76) Inventors: Ernest Newton Sumrall, Houston, TX (US); John A. Scialdone, Diamondhead, MS (US)

Correspondence Address: DICKINSON WRIGHT PLLC, 1901 L. STREET NW, SUITE 800, WASHINGTON, DC, 20036, US

(21) Appl. No.: 11/763,869

(22) Filed: Jun. 15, 2007

Related U.S. Application Data
(63) Continuation-in-part of application No. 11/014,964, filed on Dec. 20, 2004, now abandoned.

(51) Int. Cl. H01J 61/38

(52) U.S. Cl. 313/112

(57) ABSTRACT

A germicidal ultra-violet lamp (UVGI Lamp) fitted with a fluoro-polymer sleeve, sealed on each end and loose over the body of the lamp, thereby providing a thermal barrier for more efficient operation of the lamp in colder temperatures. Specifically, the invention is an insulated germicidal ultra-violet lamp (UVGI Lamp), a thermally insulative and UV transmissive sleeve (such as a fluoro-polymer like PTFE or FEP) of greater diameter than the translucent body disposed coaxially with the translucent body forming an encapsulated space therebetween filled filled with gas, such as ambient surface air. The cathodes can be UVc emitters and can UV light <255 nm. An additional feature of the present invention can be a spacer disposed within said space, wherein the distance between said translucent body and said sleeve is maintained.

### RELATED APPLICATIONS

This application is a continuation-in-part (CIP) of U.S. Non-Provisional application Ser. No. 11/014,964, filed on Dec. 20, 2004 (which claimed benefit of U.S. Provisional Application No. 60/514,590 filed Oct. 27, 2003), currently pending, and claims benefit of this earlier filing date under 35 U.S.C. §120, the contents of which are herein incorporated by reference to the extent allowed by law.

### FIELD OF INVENTION

The present invention generally relates to a germicidal ultra-violet lamp (UVGI Lamp) and, specifically, to a UVGI lamp fitted with a fluoro-polymer sleeve, which is heat sealed on each end of the lamp and loose over the body of the lamp, thereby providing a thermal barrier for more efficient operation of the lamp in colder temperatures.

### BACKGROUND OF THE INVENTION

UVGI Lamps are known in the art. For example, U.S. Pat. No. 6,193,894 to Hollander discloses a specific submersible lamp design having a fluoro-polymer protective shield. This protective shield may be shrink-wrapped on the surface of the lamp or configured to have a fluid dynamic characteristic to encapsulate the lamp in a fluid containing device, such as a water cooler (col. 5, lines 13-29). There is no intended space between the lamp and the protective sleeve for insulative purposes. In fact, the design is limited to protect the lamp from breaking or contaminating the fluid with, for example, glass fragments should the lamp break. When submersed, the protective sleeve would be forced adjacent to the lamp from the compressive force of the fluid.

Also know in the art is U.S. Pat. No. 6,057,635 to Nishimura. Nishimura teaches a method of manufacture and device which involves the use of glass or rigid material to contain a vacuum (or near vacuum) as an outer tube to the arc tube. Since the invention has a double-tube structure, a stated advantage is that it is about twice as strong as a structure that omits this outer tube. Nishimura teaches that this near vacuum reduces the need for larger diameter tubes in order to obtain greater heat retaining capacity of a lamp. Ideally, the space between the inner and outer tuber should be less than 8 mm. Most effectively, this device is intended for use with a mercury vapor lamp.

Unfortunately, there is no known device in the art to address an inexpensive application of a fluoro-polymer sleeve to maximize thermal insulation and UV light emission in atmospheric environments.

### SUMMARY OF THE INVENTION

The present invention generally relates to a germicidal ultra-violet lamp (UVGI Lamp) fitted with a fluoro-polymer sleeve, which is heat sealed on each end of the lamp and loose over the body of the lamp, thereby providing a thermal barrier for more efficient operation of the lamp in colder temperatures.

Specifically, the invention is an insulated germicidal ultra-violet lamp (UVGI Lamp) having a translucent body, hot cathodes sealed in the body, the body having first and second sealed end caps, a thermally insulative and UV transmissive sleeve of greater diameter than the translucent body disposed coaxially with the translucent body forming a space therebetween, the space being at least 8.1 mm between the translucent body and the sleeve, the sleeve attached to the translucent body in an air tight seal at or near the end caps; and the space filled with gas.

Other specific features are included. The preferred gas is ambient surface air, and can be at 101 kPa. The cathodes can be UVc emitters and can emit UV light <255 nm.

The sleeve can be a fluoro-polymer, such as PTFE or FEP.

An additional feature of the present invention can be a spacer disposed within said space, wherein the distance between the translucent body and the sleeve is maintained.

Additional aspects and advantages of the invention will become apparent from the following detailed description, the drawings, and the appended claims.

### BRIEF DESCRIPTION OF THE FIGURES

The foregoing features, as well as other features, will become apparent with reference to the description and figures below, in which like numerals represent like elements, and in which:

FIG. 1 is a diagram of the manufacturing process of one embodiment of the present invention UVGI.

FIG. 2 shows a working UGVI model fitted with fluoro-polymer sleeve.

FIG. 3 is a diagram of a prior art UVGI lamp ceramic end cap without the fluoro-polymer sleeve of the present invention.

FIG. 4 is a diagram of a test result demonstrating improved output capacity when using the present invention UVGI sleeve.

FIG. 5 provides an additional diagram of a test result demonstrating improved output capacity when using the present invention UVGI sleeve.

### DETAILED DESCRIPTION OF PREFERRED EMBODIMENTS

The present invention relates to a germicidal ultra-violet lamp (UVGI Lamp) fitted with a fluoro-polymer sleeve, which is heat sealed on each end of the lamp and loose over the body of the lamp, thereby providing a thermal barrier for more efficient operation of the lamp in colder temperatures. It is noted that throughout the specification, use of the term fluoro-polymer is for illustrative purposes only and may also include such fluoro-polymers as those sold under the trade names of TEFLON and TEFZEL, PFE, PTFE, FEP, PFA, AF, ETFE, and the like. In addition, some silicon based materials or other UV transmissive materials can be used for the sleeve.

UVGIs have been used for disinfecting surface and airborne mold and bacteria within the line of sight of the light emission of a device and typically placed in air handlers, air conditioning units, air purifiers, food storage or processing, or other devices used to move, transport, or purify air or water/liquids. A disadvantage of these types of applications is the reduced output of the UVGI in these colder environments. Lower temperature air degrades the efficiency of the UVGI.

The present invention shrink wraps a fluoro-polymer sleeve at opposite ends of the UVGI lamp, sealing both ends of the lamp, thus creating a gas insulation barrier of the remaining length of the lamp between the surface of the UVGI and the fluoro-polymer sleeve. This is a significant improvement over the current technology, which utilizes a substantially more expensive and cost prohibitive quartz sleeve or heat shrinking a fluoro-polymer sleeve the entire length of the light emitting surface of the UVGI lamp to accomplish this insulating characteristic. Instead, the present invention shrink wraps the fluoro-polymer sleeve at opposite ends of the UVGI lamp to make an airtight seal, thereby creating an insulation barrier over the remaining length of the lamp between the surface of the UVGI and the exterior surface of the fluoro-polymer sleeve.

Generally, the process to assemble the device of the present invention involves positioning; for example, an FEP or PTFE fluoro-polymer sleeve over the top of the existing UVGI lamp, cutting the shield to length and applying heat over the length of and on the edge of the ceramic surface on each end of the lamp, forming a seal over the end sections. The end result of the process described involves a UVGI lamp covered with a fluoro-polymer shield, sealed at each end of the ceramic cap and/or additionally sealed at the edges of the ceramic cap, such that the fluoro-polymer shield is in contact with and sealing the ends of the lamp, but not in contact and, instead, maintaining an air barrier around the surface of the UVGI Lamp. The air barrier thereby creates insulation around the lamp, allowing it to be more resistant to temperature change and allowing the lamp to operate more efficiently. This device further allows for significant reduction in manufacturing costs than that available under the current technology.

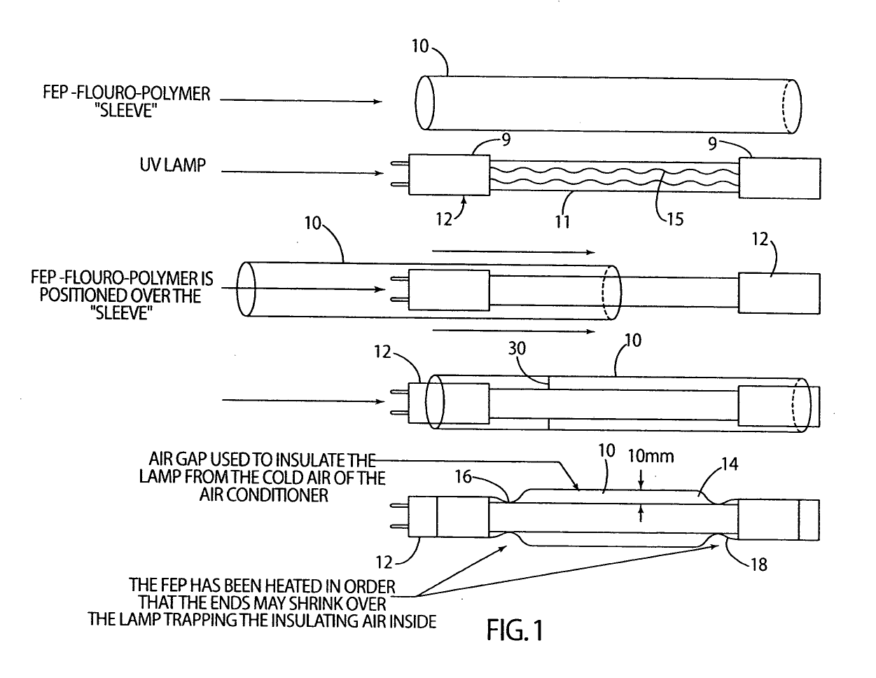

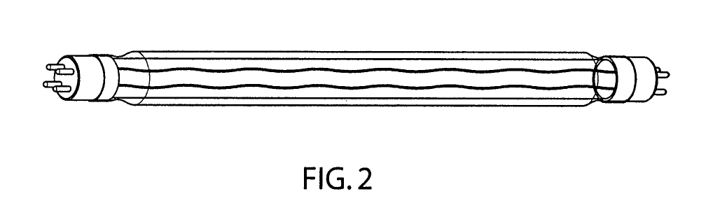

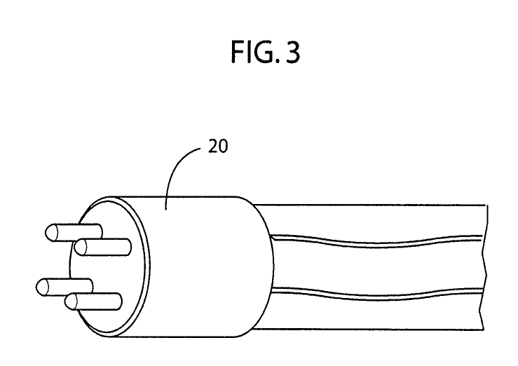

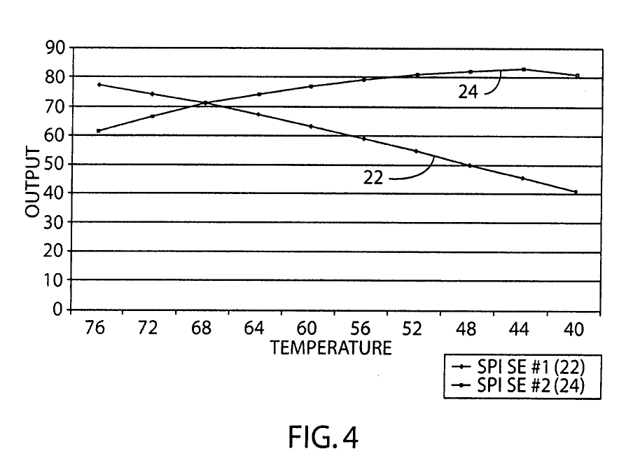

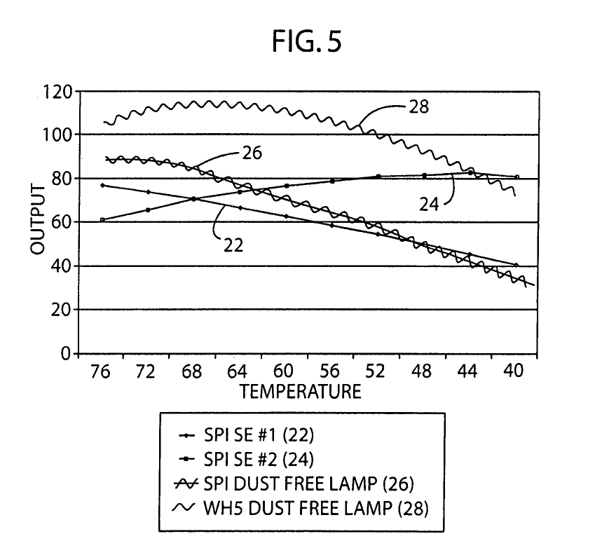

Referring now to the figures, FIG. 1 is a diagram of a manufacturing process of one embodiment of the present invention. As shown in the illustration, a fluoro-polymer sleeve 10 is positioned over a UV lamp (generally shown at 12) having a smaller diameter than sleeve 10. UV Lamp 12 as shown has two end caps 9, a translucent tube 11, and hot cathodes (described below) 15. Sleeve 10 is thus disposed coaxially with UV lamp 12, forming a space and occupied by a Gas 14 therebetween. The preferred difference in diameter is greater than 8 mm and can approximates 10 mm. It is also noted that use of the term gas can mean air or any fraction or equivalent thereof, or any other gas, such as an inert gas, that can have insulative properties with no or minimal reduction in UV transmission. In use, Gas 14 insulates UV lamp 14 from the colder air of, for example, an air conditioner and/or other median. Gas 14 is encapsulated within the space defined between Sleeve 10 and UV lamp 12 through a heat shrinking process at First End 16 and Second End 18. As demonstrated in FIGS. 4 and 5 (discussed below), the present invention dramatically increases the output of the UVGI compared to a control UGVI without Sleeve 10 installed. FIG. 2 shows a completed device, and FIG. 3 shows a typical prior art UVGI ceramic cap 20, though these end caps may be made from many other suitable materials, such as plastic.

Referring now to FIG. 4, SPI SE #1 (22) illustrates the UV output of a UVGI Lamp as a function of temperature where the lamp has a prior art polymer coating over its entire length such as described in U.S. Pat. No. 6,193,894 to Hollander. As shown, the polymer does provide minimal thermal protection to UV output. In contrast, SPI SE #2 (24) shows the same type of UVGI fitted with the fluoro-polymer sleeve 10 fixture of the present invention, including Gas 14 as described above. As previously described, the lamp was hermetically sealed on the end caps and loose over the body of the lamp. This forms an insulating layer of air which, as shown in the figure, effectively doubled the output of the device.

Referring to FIG. 5, two other UGVI Lamps, having no polymer or encapsulated air insulation, were tested for UV output as a function of temperature. The SPI Dust Free lamp (26) shows UV output of a UVGI using a ballast, sold under the name WORK HORSE 2, and a 24″ lamp. As illustrated, at first, the un-encapsulated UVGI output (26) is more efficient. But as temperature drops, as one would see in an air conditioning system, its efficiency also drops. SPI SE #1 (22) has increased efficiency only at around 48 degrees, though the effect is slight.

Still referring to FIG. 5, WH5 Dust free lamp (28) refers to a UVGI fixture with a ballast, sold under the trade name WORK HORSE 5. This ballast uses double the energy as the WORK HORSE 2, as used in the UGVI shown in SPI Dust Free lamp (26) and SPI SE #2 (26). Nevertheless, using the present invention, the same output is achieved at 40 degrees at half the energy. Basically, the tests show that even with the ½ power input, the present invention doubles the output of UV energy.

Thus, the present invention can effectively and efficiently sterilize air, water, and surfaces using UVGI. It is noted that UGVI Lamps typically have an ultraviolet light at 254 nm and lower using hot cathode bulbs. The device of the present invention can be made of any industry standard ultraviolet lamp using hot cathode technology having electrodes on one or both ends and producing ultraviolet light in the UVc germicidal range, which is 254 nm and lower. The inner surface of Sleeve 10 is preferably at least 8 mm distant from the outer surface of the lamp and may be much more. The fluoro-polymer must be pliable enough to be hermetically sealed on the ends with heat, but rigid enough to maintain a nearly uniform air gap over the body of the bulb with a gap of at least 8 mm, but preferably more. The fluoro-polymer tube is heat shrunk to the ends of the bulb and the caps. The gas contained within the tube may be, but is neither limited to nor confined by, air at approximately 101 kPa in pressure.

The result of the present invention is to stabilize the hot cathode bulb by stabilizing the temperature of the medium to which the lamp may be exposed. The medium trapped within the fluoro-polymer tube is preferably filtered surface air. This method nearly doubles the efficiency of UVc lamps used in HVAC systems in the summer. The fluoro-polymer sleeve must only be in contact with the bulb at the ends or caps and may only contact optional Spacers 30 used in the middle of the lamp, for example, that help maintain the nearly uniform gap. The efficiency of the ultraviolet lamp is achieved by improving the temperature to which the lamp is exposed. The thermally insulating layer of air is heated by the lamp and cooled by the exterior fluid flow. It provides a very efficient buffer, which increases the efficiency of the hot cathode lamp. The fluoro-polymer tubing is hermetically sealed at the ends/caps of the ultraviolet bulb.

These features of the present invention are distinct from prior art devices in that the present invention is specifically concerned with improving the efficiency of a device that emits electromagnetic energy in the UV spectrum, specifically 253.7 nm and lower, which is referred to as UVc in the industry. Using a glass sleeve for a lamp intended to emit 253.7 nm and lower wavelengths of energy is not workable since glass filters out UVc and would, therefore, be of little or no use in the creation of a device for this purpose. Also, the present invention does not require the sleeve to be rigid enough to allow the creation of a near vacuum condition, as doing so would negate the effective usefulness of PTFE or other similar fluoro-polymer materials that may be used to be heated and shrink fit onto the ends of the lamps. PTFE is preferable for use in the present invention because it is cost effective and pliable. A vacuum would actually pull the fluoro-polymer tube into contact with the UVGI, which would negate the effectiveness of the insulating air pocket between the outer tube of the UVc emitter and the sleeve.

Additionally, other prior art inventions, such as those described in U.S. Pat. No. 6,057,635 to Nishimura, make use of phosphor as a coating to the inner tube, giving a clear indication of the specific nature of the invention, which is to provide energy in the spectrum visible to Humans. In contrast, the present invention is not concerned with luminance, but with the creation and emittance of electromagnetic radiation at ˜254 nm and lower for the purposes of disinfection of air, water, and surfaces. Any visible light created by a lamp producing UVc energy is an unfortunate and inefficient byproduct of the production of the ionizing energy wavelength of 253.7 nm and lower. Thus, the present invention is not concerned with visible light, but is concerned with increased efficiency. In short, the addition of the fluoro-polymer and sleeve combination of HOT CATHODE lamps and ambient pressure air gaps between the lamp and the sleeve allow nearly 100 percent increase in efficiency.

While the invention has been described in conjunction with specific embodiments, it is evident that many alternatives, modifications, and variations will be apparent to those skilled in the art in light of the foregoing description. Accordingly, the present invention attempts to embrace all such alternatives, modifications, and variations that fall within the spirit and scope of the appended claims.

The invention claimed is:

1. An insulated germicidal ultra-violet lamp (UVGI Lamp), comprising:
a translucent body;
hot cathodes sealed in said body;
said body having first and second sealed end caps;
a thermally insulative and UV transmissive sleeve of greater diameter than said translucent body disposed coaxially with the translucent body forming a space therebetween;
said space being at least 8.1 mm between said translucent body and said sleeve;
said sleeve attached to said translucent body in an air tight seal at or near said end caps; and
said space filled with gas.

2. The lamp of claim 1, wherein said gas is filtered surface air.

3. The lamp of claim 2, wherein said air is 101 kPa.

4. The lamp of claim 1, wherein said cathodes are UVc emitters.

5. The lamp of claim 4, wherein said cathodes emit UV light <255 nm.

6. The lamp of claim 1, wherein said sleeve is a fluoro-polymer.

7. The lamp of claim 6, wherein said fluoro-polymer is PTFE.

8. The lamp of claim 6, wherein said fluor-polymer is FEP.

9. The lamp of claim 1, wherein said space is 10 mm.

10. The lamp of claim 1, further comprising a spacer disposed within said space, wherein the distance between said translucent body and said sleeve is maintained.

11. The lamp of claim 7, wherein said sleeve is attached to said translucent body in an airtight seal by heat shrinking.

12. An insulated germicidal ultra-violet lamp (UVGI Lamp), comprising:
a translucent body;
hot cathodes sealed in said body emitting a UVc of <255 nm;
said body having first and second sealed end caps;
a thermally insulative and UV transmissive fluoro-polymer PTFE sleeve of at least 16.1 mm diameter than said translucent body disposed coaxially with the translucent body forming a space therebetween;
said sleeve attached to said translucent body in an air tight seal at or near said end caps; and
said space filled with ambient surface air.

13. A germicidal ultra-violet lamp (UVGI Lamp) fitted with a fluoro-polymer sleeve, which is heat sealed on each end of the lamp but is left loose over the body of the lamp, thereby providing an air barrier between the glass, quartz, or other surface of the lamp and the fluoro-polymer sleeve for more efficient operation of the lamp in colder temperatures.
